(19) United States
(12) Patent Application Publication
(10) Pub. No.: US 2005/0208004 A1
(43) Pub. Date: Sep. 22, 2005

(54) Ionic blends and methods of preparation and application thereof

(76) Inventors: Fernando Romero, Los Angeles, CA (US); Kay Askew Romero, Los Angeles, CA (US)

Correspondence Address: CHRISTIE, PARKER & HALE, LLP, PO BOX 7068, PASADENA, CA, 91109-7068, US

(21) Appl. No.: 10/804,287

(22) Filed: Mar. 18, 2004

(51) Int. Cl. A61K 7/025; A61K 7/06

(52) U.S. Cl. 424/70.1

(57) ABSTRACT

A mineral composition for use in hair care products is provided. The composition comprises a powder of at least one poly-element mineral which may be suspended in solution with a compatible solvent, preferably deionized water or glycol.

### FIELD OF THE INVENTION

The present invention relates to mineral compositions for use in hair care products and to methods for their preparation and application.

### BACKGROUND OF THE INVENTION

Damage is routinely done to the hair upon application of heat from styling implements such as hairdryers and curling irons. In addition to damage caused by the use of heat to style the hair, other hair care products, such as shampoo, conditioner and various hairsprays, gels and mousses, can be harsh on the hair and the scalp. Consequently, repeated use of these products on a daily basis can damage the hair and scalp. The brushing or combing of the hair after shampooing can also cause damage to the hair, such as split ends, leaving the hair dry and brittle, and adversely affecting the appearance of the hair. In addition, when hair sprays, gels and/or mousses are used in combination with heat from a hair dryer or curling iron, the damage to the hair can be increased.

Shampooing the hair strips the hair and scalp of its natural minerals, often leaving the hair looking dry and brittle, and making it very difficult to comb or brush. The use of conditioner after shampooing the hair is intended to repair some of the damage caused by the shampooing. However, the conditioner is sometimes difficult to rinse out of the hair, thereby leaving the hair with a greasy texture that adversely affects the appearance of the hair by reducing the shiny appearance of the hair.

However, shampooing the hair is necessary to clean out the sebum secreted by the scalp and the dirt that accumulates in the hair. Also, conditioning is preferred in order to make the hair easier to comb or brush and facilitate easier styling of the hair. Styling of the hair often requires the use of both heat from a hair dryer or curling iron, and styling products such as hairspray, gel or mousse. This combination, however, causes damage to the hair adversely affecting its appearance. Therefore, a need arises for hair care products that are less damaging to the hair and scalp, but that retain their intrinsic qualities.

Poly-element minerals, such as tourmaline, perlite and pitchstone, in combination with other materials, have been suggested for use in connection with the hair and scalp. In U.S. Pat. No. 6,357,075 B1 to Kaizuka, a hair brush is disclosed having bristles containing poly-element minerals and infrared radiation materials. The poly-element minerals are used along with the infrared radiation materials, such as alumina or titania, to coat the bristles of the hair brush. The infrared radiation materials are stated to generate extreme infrared radiation with wavelengths that are easily absorbed into the hair and scalp. The poly-element minerals are said to generate electromagnetic waves that electrically transform the nuclei of the infrared radiation materials, causing the atoms of those materials to reach excited states. This electrical transformation allegedly causes the water within the hair to be mineralized, and the protein in the scalp to be activated.

Also, in U.S. Pat. No. 6,205,674 B1 to Kaizuka, a hair dryer is provided having a ceramic plate coated with a powder of both poly-element minerals and extreme infrared radiation materials. The interaction between the poly-element minerals and the extreme infrared radiation materials is described in the same manner as in U.S. Pat. No. 6,357,075 B1, described above. When the air from the hairdryer passes over the coated ceramic plate, the electromagnetic waves generated from the poly-element minerals are allegedly blown onto the wet hair, thereby mineralizing the water within the hair, and activating the protein in the hair and scalp.

Finally, in U.S. Pat. No. 5,941,253 also to Kaizuka, a curling iron is disclosed having a powder of both poly-element minerals and fluoroplastic coated on the surface. The poly-element minerals are said to generate negative ions radiating from the surface of the curling iron, thereby shortening the polymerization of water molecules and sufficiently impregnating the hair with water. The negative ions emanating from the surface of the curling iron allegedly activate the protein within the hair and scalp.

In the above cited patents, the poly-element minerals are always used along with either fluoroplastic or extreme infrared radiation materials. It is asserted to be the generation of electromagnetic waves or negative ions from the poly-element minerals that effects the mineralization of the water within the hair and the activation of protein within the hair and scalp. Moreover, in each of these patents, the poly-element minerals are brought in contact with the hair or scalp during only certain phases or aspects of hair care, i.e., brushing, drying or curling. Thus, contact can only occur during certain very specific activities that may or may not be used by any particular person at any particular time. The modes of contact are also somewhat tenuous such that intimate and comprehensive contact with the hair and scalp is not achieved.

In U.S. Pat. No. 6,036,965 to Gubernick et al., cosmetic skin or hair cleansing compositions comprising tourmaline are described. The compositions are formulated and used for cleansing.

### SUMMARY OF THE INVENTION

The present invention is directed in part to a mineral composition for use in hair care products to enhance the performance of those products and to augment the look and feel of the hair. In one embodiment, the mineral composition comprises a powder of at least one poly-element mineral selected from the group consisting of perlite, tourmaline and pitchstone. Any combination of these poly-element minerals may also be used. In another embodiment, the mineral composition comprises at least one poly-element mineral suspended in a compatible solvent. Preferred solvents for use in this embodiment are deionized water and glycol or its derivatives. In a third embodiment, the mineral composition comprises an ionic mineral extract. One method for the preparation of the ionic extract of poly-element mineral and solvent comprises suspending the poly-element mineral in a compatible solvent and heating the resulting solution under pressure for a suitable length of time. A mineral composition according to any of these embodiments may be applied to the hair by first thoroughly mixing it into a hair care product then applying the hair care product to the hair.

The use of poly-element minerals in hair care products exhibits enhancements in several areas. The minerals impart a better fragrance to the products and to the hair after use. The minerals also make the hair easier to comb after drying. The look and feel of the hair is also enhanced, including a reduction in static flyaways and increased sheen and luster. In general, the overall condition of the hair is enhanced.

Certain embodiments of the poly-element mineral composition disclosed herein, when added to hair care products will decrease damage done to the hair by those products. Certain embodiments of the poly-element mineral composition will also enhance the performance of hair care products, thereby improving the look and feel of the hair.

### DETAILED DESCRIPTION OF THE INVENTION

In one embodiment of the present invention, a mineral composition is provided for use in hair care products. Hair care products include shampoos, conditioners, hair sprays, hair gels, permanent wave relaxers, hair structure altering preparations, hair colors, hair bleaches, and any other hair care products. The composition comprises a powder of at least one poly-element mineral selected from the group consisting of tourmaline, perlite and pitchstone. The powder is preferably prepared by grinding (or milling) the at least one poly-element mineral to a particle size ranging from about 0.5 μm to about 25 μm. Preferably, the at least one poly-element mineral is ground to a powder having a particle size ranging from about 0.5 μm to about 10 μm. More preferably, the at least one poly-element mineral is ground to a powder having a particle size ranging from about 0.5 μm to about 5.0 μm. Even more preferably, the at least one poly-element mineral is ground to a powder having a particle size ranging from about 0.5 μm to about 2.5 μm. Still more preferably, the at least one poly-element mineral is ground to a powder having a particle size ranging from about 0.5 μm to about 1.0 μm. The powder may consist of only one poly-element mineral, or may consist of any combination of two or three of the poly-element minerals. Preferably, a blend of at least two poly-element minerals is used. The preferred blend of minerals for addition into hair care products comprises tourmaline and perlite.

When the powder comprises a combination of poly-element minerals, each poly-element mineral may be present in any amount. However, when the powder comprises a combination of tourmaline and perlite, tourmaline is generally present in an amount ranging from about 0.5% to about 99.5% by weight, with perlite making up the remaining wt %. Preferably, tourmaline is present in an amount ranging from about 0.5% to about 50% by weight, with perlite making up the remaining wt %. More preferably, tourmaline is present in amount of about 0.5% by weight, with perlite making up the remaining 99.5% by weight.

When the powder comprises a combination of tourmaline and pitchstone, tourmaline is generally present in an amount ranging from about 0.5% to about 99.5% by weight, with pitchstone making up the remaining wt %. Preferably, tourmaline is present in an amount ranging from about 0.5% to about 50% by weight, with pitchstone making up the remaining wt %. More preferably, tourmaline is present in amount of about 0.5% by weight, with pitchstone making up the remaining 99.5% by weight.

When the powder comprises a combination of perlite and pitchstone, perlite is generally present in an amount ranging from about 0.5% to about 99.5% by weight, with pitchstone making up the remaining wt %. Preferably, perlite is present in an amount ranging from about 0.5% to about 50% by weight, with pitchstone making up the remaining wt %. More preferably, perlite is present in amount of about 0.5% by weight, with pitchstone making up the remaining 99.5% by weight.

When the powder comprises a combination of all three poly-element minerals, tourmaline is generally present in an amount ranging from about 0.1% to about 99.8% by weight, and perlite is generally present in an amount ranging from about 0.1% to about 99.8% by weight, with pitchstone making up the remaining wt %. In one aspect, tourmaline is present in an amount of about 99.8% by weight, perlite is present in an amount of about 0.1%, and pitchstone is present in an amount of about 0.1% by weight. In a second aspect, tourmaline is present in an amount of about 0.1% by weight, perlite is present in an amount of about 99.8% by weight, and pitchstone is present in an amount of about 0.1% by weight. In an alternative aspect, tourmaline is present in an amount of about 0.1% by weight, perlite is present in an amount of about 0.1% by weight, and pitchstone is present in an amount of about 99.8% by weight.

In an alternative embodiment, a suspended solution is prepared by suspending a powder of the poly-element minerals in a compatible solvent. The solution comprises a powder of poly-element minerals, prepared as described above, suspended in a compatible solvent. The solution is prepared by mixing the solution until the powder of poly-element minerals is completely suspended in the compatible solvent. As above, the poly-element minerals are ground to a powder having a particle size ranging from about 0.5 μm to about 25 μm. Preferably, the poly-element minerals are ground to a powder having a particle size ranging from about 0.5 μm to about 10 μm. More preferably, the poly-element minerals are ground to a powder having a particle size ranging from about 0.5 μm to about 5.0 μm. Even more preferably, the poly-element minerals are ground to a powder having a particle size ranging from about 0.5 μm to about 2.5 μm. Still more preferably, the poly-element minerals are ground to a powder having a particle size ranging from about 0.5 μm to about 1.0 μm. The powder may consist of only one poly-element mineral, or may consist of any combination of two or three of the poly-element minerals. Preferably, a blend of at least two poly-element minerals is used. The preferred blend of minerals comprises tourmaline and perlite.

In a third embodiment, an ionic mineral extract is prepared by suspending a powder of the poly-element minerals in a solution and exposing the resulting solution to heat and pressure. The heated suspension of the poly-element minerals in the compatible solvent under pressure causes the extraction of mineral ions into the compatible solvent. The suspension of these ions in the compatible solvent creates an ionic mineral extract. Upon completion of the extraction process, described in more detail below, any remaining powder particles are removed from the solvent, and only the ionic mineral extract is then added to the hair care product.

The solution used in the extraction process comprises a powder of poly-element minerals, prepared substantially as above, suspended in a compatible solvent. In this embodiment, the particle size of the powder that is suspended in the compatible solvent can be substantially greater than the particle size of the powder that is directly added into the hair care product because any remaining powder is removed from the ionic extract. The powder used for this embodiment is prepared by grinding the at least one poly-element mineral to a powder having a particle size generally ranging from about 0.05 mm to about 25 mm. Preferably, the at least one poly-element mineral is ground to a powder having a particle size ranging from 0.05 mm to about 10 mm. More preferably, the at least one poly-element mineral is ground to a powder having a particle size ranging from about 0.05 mm to about 5 mm. Even more preferably, the at least one poly-element mineral is ground to a powder having a particle size ranging from about 0.05 mm to about 0.10 mm. The powder may consist of only one poly-element mineral, or may consist of any combination of two or three of the poly-element minerals. Preferably, a blend of at least two poly-element minerals is used. The preferred blend of minerals comprises tourmaline and perlite.

A “compatible solvent” is a solvent that is relatively non-toxic and able to suspend the particles of the mineral composition. The solvent may be selected from the group consisting of deionized water, glycol and its derivatives, organic acids and bases and their derivatives, inorganic acids and bases and their derivatives, organic amines, and salt solutions. Preferably, the compatible solvent is selected from the group consisting of deionized water, glycol and glycol derivatives. In one embodiment, glycol or its derivatives are used, wherein glycol derivatives are selected from the group consisting of propylene glycol, hexylene glycol, ethylene glycol, diethylene glycol, and glycerin. In another embodiment, deionized water is used. When preparing a suspended solution, the weight ratio of the poly-element mineral powder to the compatible solvent preferably ranges from about 1:5 to about 1:20. More preferably, the weight ratio of the poly-element mineral powder to the compatible solvent ranges from about 1:5 to about 1:10. When preparing an ionic mineral extract, the weight ratio of the poly-element mineral powder to the compatible solvent preferably ranges from about 0.05:1 to about 2.5:1. More preferably, the weight ratio of the poly-element mineral powder to the compatible solvent ranges from 0.5:1 to 1:1.

One method for preparing the ionic mineral extract comprises suspending the powder in the solvent, as above, and heating the suspended solution under pressure for a suitable period of time. A “suitable period of time” is any length of time sufficient to cause the extraction of mineral ions from the poly-element mineral powder into the compatible solvent. Time periods in the range of 12 to 120 hours are usually sufficient. When the ionic mineral extract is added to a hair care product, the ions in the ionic mineral extract react with the ingredients of the hair care product, thereby enhancing the performance of those products upon application to the hair.

In a particularly preferred embodiment, the poly-element mineral powder is suspended in a solution of deionized water and heated under pressure for a suitable length of time to cause the extraction of mineral ions into the deionized water. In this embodiment, the suspended solution is preferably heated at a temperature ranging from about 5° C. to about 100° C. More preferably, the suspended solution is heated at a temperature ranging from about 30° C. to about 80° C. Even more preferably, the suspended solution is heated at a temperature ranging from about 50° C. to about 60° C. Preferably, the suspended solution is heated under a pressure ranging from about 1 atm to about 5 atm. More preferably, the suspended solution is heated under a pressure ranging from about 1 atm to about 2 atm. Even more preferably, the suspended solution is heated under a pressure ranging from about 1 atm to about 1.25 atm. Preferably, the suspended solution is heated under pressure for a period of time ranging from about 12 hours to about 120 hours. More preferably, the suspended solution is heated under pressure for a period of time ranging from about 24 to about 48 hours.

An alternative method for preparing the ionic mineral extract comprises suspending the poly-element mineral powder in a solution of glycol or one of its derivatives and heated under pressure for a suitable length of time to cause the extraction of mineral ions into the glycol. In this embodiment, the suspended solution may be heated at a temperature ranging from about 75° C. to about 200° C. Preferably, the suspended solution is heated at a temperature ranging from about 150° C. to about 200° C. Alternatively, the suspended solution is heated at a temperature ranging from about 100° C. to about 150° C. In another alternative, the suspended solution is heated at a temperature ranging from about 75° C. to about 100° C. Preferably, the suspended solution is heated under a pressure ranging from about 1 atm to about 5 atm. More preferably, the suspended solution is heated under a pressure ranging from about 1 atm to about 2 atm. Even more preferably, the suspended solution is heated under a pressure ranging from about 1 atm to about 1.25 atm. Preferably, the suspended solution is heated under pressure for a period of time ranging from about 12 hours to about 120 hours. More preferably, the suspended solution is heated under pressure for a period of time ranging from about 24 hours to about 48 hours.

To apply the mineral composition to the hair, it is preferably first added to a hair care product. When the mineral composition comprises a powder of poly-element minerals, the powder is added to the hair care product in an amount ranging from about 0.01% to about 10% by weight. Preferably, the powder is added to the hair care product in an amount ranging from about 0.01% to about 5% by weight. More preferably, the powder is added to the hair care product in an amount ranging from about 0.01% to about 0.5% by weight. Even more preferably, the powder is added to the hair care product in an amount ranging from about 0.01% to about 0.10% by weight.

When the mineral composition comprises a suspended solution, the solution is added to the hair care product in an amount ranging from about 0.01% to about 10% by weight. Preferably, the solution is added to the hair care product in an amount ranging from about 0.01% to about 5% by weight. More preferably, the solution is added to the hair care product in an amount ranging from about 0.01% to about 0.5% by weight. Even more preferably, the powder is added to the hair care product in an amount ranging from about 0.01% to about 0.10% by weight.

When the mineral composition comprises an ionic mineral extract, the extract is added to the hair care product in an amount ranging from about 0.01% to about 10% by weight. Preferably, the extract is added to the hair care product in an amount ranging from about 0.01% to about 5% by weight. More preferably, the extract is added to the hair care product in an amount ranging from about 0.01% to about 0.5% by weight. Even more preferably, the extract is added to the hair care product in an amount ranging from about 0.01% to about 0.10% by weight.

After the mineral composition is added to the hair care product, it is thoroughly mixed into the product. After mixing, the hair care product is applied to the hair according to the product's instructions. The presently preferred mineral composition is the ionic mineral extract because the extract requires only minimal mixing when added to the hair care product. The suspended solution is less preferable because it requires the removal of solvent from the hair care product in order to compensate for the addition of solvent from the solution. The removal of solvent is necessary when adding a suspended solution to the hair care product in order to maintain the original consistency of the hair care product. Also, the solution requires more mixing than the extract. The powder is also less preferable because it requires considerably more mixing than either the solution or the extract in order to uniformly distribute the powder throughout the hair care product to which it is added. In addition, the density of the poly-element minerals will eventually cause them to separate from solution. Accordingly, the poly-element minerals, whether in powder form or in solution, will eventually separate from the hair care product. However, the extract does not include solid mineral particles, and is therefore preferable because it does not include solid particles that can separate from the solution.

Performance Test Methods

A mineral composition prepared by heating under pressure a suspended solution of perlite powder in deionized water was added to three commercially available shampoos, three commercially available conditioners, and three commercially available hairsprays. The products with the mineral composition added were tested alongside the same products without the mineral composition. Each product, both with and without the composition, was rated separately by eleven professional hairstylists. Each stylist tested each product by applying the product containing the mineral composition on one half of his head, and applying the product without the composition on the other half of his head. Tables 1 through 6 summarize the ratings given by the eleven hairstylists to each shampoo, both with and without the mineral composition. The hairstylists rated each shampoo according to several characteristics on a scale of 1 to 10, 10 being the best rating. The shampoos containing the mineral composition exhibited enhanced performance. These shampoos imparted an enhanced gloss, enhanced conditioning and moisturizing, reduced the amount of static flyaways, and enhanced the ease of combing and brushing.

TABLE 1
Shampoo A Without Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 8 | 4 | 10 | 10 | 5 | 7 | 5 | 10 | 8 | 5 | 8 | 7.27 |
| Product | 8 | 7 | 5 | 10 | 10 | 7 | 5 | 9 | 5 | 5 | 5 | 6.91 |
| Fragrance | | | | | | | | | | | | |
| Pourability | 6 | 6 | 10 | 4 | 9 | 9 | 4 | 9 | 10 | 8 | 9 | 7.64 |
| Application | 7 | 4 | 10 | 8 | 9 | 6 | 10 | 6 | 6 | 8 | 9 | 7.55 |
| Ease | | | | | | | | | | | | |
| Fragrance at | 5 | 7 | 6 | 9 | 9 | 10 | 10 | 8 | 4 | 6 | 7 | 7.36 |
| Application | | | | | | | | | | | | |
| Foam Quality | 10 | 5 | 10 | 7 | 8 | 10 | 4 | 10 | 5 | 6 | 8 | 7.55 |
| Foam | 7 | 7 | 8 | 4 | 10 | 7 | 7 | 6 | 6 | 10 | 4 | 6.91 |
| Quantity | | | | | | | | | | | | |
| Fragrance | 7 | 5 | 4 | 1 | 2 | 6 | 4 | 5 | 4 | 1 | 1 | 3.64 |
| during | | | | | | | | | | | | |
| Drying | | | | | | | | | | | | |
| Ease of Wet | 2 | 2 | 5 | 7 | 5 | 2 | 4 | 2 | 5 | 2 | 3 | 3.55 |
| Combability | | | | | | | | | | | | |
| Ease of Dry | 5 | 2 | 7 | 2 | 5 | 4 | 2 | 5 | 6 | 4 | 7 | 4.45 |
| Combability | | | | | | | | | | | | |
| Residual | 6 | 3 | 7 | 4 | 1 | 6 | 6 | 5 | 2 | 1 | 4 | 4.09 |
| Fragrance on | | | | | | | | | | | | |
| Hair | | | | | | | | | | | | |
| Hair Sheen | 1 | 7 | 2 | 3 | 4 | 2 | 3 | 1 | 2 | 2 | 4 | 2.82 |
| and Luster | | | | | | | | | | | | |
| Static | 6 | 7 | 1 | 2 | 2 | 7 | 4 | 6 | 5 | 3 | 6 | 4.45 |
| Flyaways | | | | | | | | | | | | |
| Feel and | 6 | 6 | 6 | 2 | 3 | 3 | 3 | 1 | 5 | 7 | 4 | 4.18 |
| Touch of Dry | | | | | | | | | | | | |
| Hair | | | | | | | | | | | | |
| Overall | 7 | 6 | 3 | 6 | 1 | 7 | 4 | 4 | 1 | 3 | 7 | 4.45 |
| Condition of | | | | | | | | | | | | |
| Hair | | | | | | | | | | | | |

TABLE 2
Shampoo A With Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 5 | 8 | 9 | 4 | 7 | 7 | 4 | 4 | 10 | 7 | 9 | 6.73 |
| Product | 10 | 9 | 9 | 6 | 7 | 8 | 10 | 10 | 6 | 8 | 7 | 8.18 |
| Fragrance | | | | | | | | | | | | |
| Pourability | 4 | 7 | 4 | 5 | 9 | 9 | 5 | 9 | 9 | 8 | 10 | 7.18 |
| Application | 5 | 6 | 4 | 4 | 9 | 4 | 9 | 9 | 8 | 4 | 7 | 6.27 |
| Ease | | | | | | | | | | | | |
| Fragrance at | 6 | 6 | 7 | 6 | 5 | 10 | 5 | 8 | 8 | 10 | 10 | 7.36 |
| Application | | | | | | | | | | | | |
| Foam Quality | 8 | 5 | 9 | 10 | 9 | 9 | 7 | 4 | 7 | 4 | 4 | 6.91 |
| Foam | 5 | 10 | 5 | 7 | 4 | 9 | 4 | 4 | 10 | 4 | 5 | 6.09 |
| Quantity | | | | | | | | | | | | |
| Fragrance | 6 | 10 | 7 | 3 | 7 | 1 | 1 | 9 | 6 | 4 | 7 | 5.55 |
| during | | | | | | | | | | | | |
| Drying | | | | | | | | | | | | |
| Ease of Wet | 1 | 3 | 1 | 1 | 6 | 1 | 1 | 2 | 9 | 4 | 9 | 3.45 |
| Combability | | | | | | | | | | | | |
| Ease of Dry | 4 | 8 | 10 | 6 | 2 | 9 | 3 | 7 | 5 | 7 | 10 | 6.45 |
| Combability | | | | | | | | | | | | |
| Residual | 1 | 7 | 10 | 4 | 7 | 2 | 7 | 10 | 7 | 5 | 8 | 6.18 |
| Fragrance on | | | | | | | | | | | | |
| Hair | | | | | | | | | | | | |
| Hair Sheen | 5 | 5 | 9 | 4 | 3 | 9 | 6 | 4 | 8 | 8 | 2 | 5.73 |
| and Luster | | | | | | | | | | | | |
| Static | 7 | 6 | 1 | 7 | 9 | 7 | 1 | 5 | 2 | 8 | 9 | 5.64 |
| Flyaways | | | | | | | | | | | | |
| Feel and | 8 | 3 | 10 | 1 | 4 | 7 | 4 | 10 | 7 | 10 | 1 | 5.91 |
| Touch of Dry | | | | | | | | | | | | |
| Hair | | | | | | | | | | | | |
| Overall | 3 | 6 | 2 | 2 | 9 | 6 | 6 | 2 | 6 | 7 | 7 | 5.09 |
| Condition of | | | | | | | | | | | | |
| Hair | | | | | | | | | | | | |

TABLE 3
Shampoo B Without Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 6 | 9 | 5 | 8 | 5 | 4 | 4 | 9 | 10 | 9 | 10 | 7.18 |
| Product | 6 | 7 | 4 | 8 | 7 | 10 | 4 | 5 | 5 | 9 | 10 | 6.82 |
| Fragrance | | | | | | | | | | | | |
| Pourability | 9 | 10 | 8 | 7 | 7 | 5 | 6 | 10 | 4 | 5 | 4 | 6.82 |
| Application | 9 | 4 | 4 | 8 | 9 | 4 | 4 | 4 | 4 | 5 | 9 | 5.82 |
| Ease | | | | | | | | | | | | |
| Fragrance at | 10 | 8 | 8 | 6 | 6 | 5 | 10 | 10 | 9 | 4 | 7 | 7.55 |
| Application | | | | | | | | | | | | |
| Foam Quality | 5 | 5 | 6 | 9 | 6 | 6 | 8 | 6 | 6 | 7 | 9 | 6.64 |
| Foam | 4 | 8 | 10 | 7 | 6 | 4 | 6 | 6 | 7 | 4 | 9 | 6.45 |
| Quantity | | | | | | | | | | | | |
| Fragrance | 5 | 5 | 6 | 7 | 3 | 6 | 6 | 1 | 4 | 7 | 4 | 4.91 |
| during | | | | | | | | | | | | |
| Drying | | | | | | | | | | | | |
| Ease of Wet | 5 | 5 | 2 | 1 | 6 | 1 | 4 | 6 | 3 | 4 | 5 | 3.82 |
| Combability | | | | | | | | | | | | |
| Ease of Dry | 6 | 4 | 2 | 4 | 3 | 4 | 1 | 4 | 6 | 4 | 7 | 4.09 |
| Combability | | | | | | | | | | | | |
| Residual | 7 | 2 | 5 | 4 | 4 | 1 | 1 | 7 | 3 | 4 | 7 | 4.09 |
| Fragrance on | | | | | | | | | | | | |
| Hair | | | | | | | | | | | | |
| Hair Sheen | 2 | 1 | 2 | 6 | 4 | 7 | 3 | 2 | 2 | 5 | 1 | 3.18 |
| and Luster | | | | | | | | | | | | |
| Static | 7 | 4 | 6 | 6 | 6 | 7 | 7 | 2 | 6 | 6 | 4 | 5.55 |
| Flyaways | | | | | | | | | | | | |
| Feel and | 5 | 3 | 7 | 6 | 5 | 3 | 3 | 3 | 5 | 1 | 6 | 4.27 |
| Touch of Dry | | | | | | | | | | | | |
| Hair | | | | | | | | | | | | |
| Overall | 4 | 2 | 5 | 2 | 6 | 4 | 7 | 3 | 7 | 3 | 4 | 4.27 |
| Condition of | | | | | | | | | | | | |
| Hair | | | | | | | | | | | | |

TABLE 4
Shampoo B With Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 8 | 10 | 9 | 5 | 5 | 7 | 4 | 10 | 7 | 4 | 4 | 6.64 |
| Product | 10 | 5 | 10 | 10 | 7 | 7 | 8 | 8 | 4 | 10 | 4 | 7.55 |
| Fragrance | | | | | | | | | | | | |
| Pourability | 8 | 4 | 4 | 8 | 8 | 7 | 8 | 4 | 4 | 5 | 5 | 5.91 |
| Application | 10 | 8 | 9 | 4 | 8 | 10 | 10 | 10 | 8 | 4 | 9 | 8.18 |
| Ease | | | | | | | | | | | | |
| Fragrance at | 6 | 9 | 4 | 8 | 7 | 4 | 9 | 7 | 9 | 5 | 4 | 6.55 |
| Application | | | | | | | | | | | | |
| Foam Quality | 5 | 9 | 6 | 4 | 5 | 4 | 7 | 9 | 4 | 4 | 10 | 6.09 |
| Foam | 8 | 8 | 9 | 7 | 5 | 10 | 8 | 10 | 4 | 8 | 10 | 7.91 |
| Quantity | | | | | | | | | | | | |
| Fragrance | 8 | 10 | 8 | 3 | 2 | 10 | 7 | 10 | 1 | 7 | 3 | 6.27 |
| during | | | | | | | | | | | | |
| Drying | | | | | | | | | | | | |
| Ease of Wet | 8 | 1 | 9 | 4 | 4 | 10 | 9 | 3 | 4 | 10 | 10 | 6.55 |
| Combability | | | | | | | | | | | | |
| Ease of Dry | 7 | 3 | 4 | 1 | 9 | 9 | 7 | 3 | 2 | 8 | 2 | 5.00 |
| Combability | | | | | | | | | | | | |
| Residual | 8 | 6 | 4 | 1 | 2 | 7 | 7 | 8 | 6 | 8 | 6 | 5.73 |
| Fragrance on | | | | | | | | | | | | |
| Hair | | | | | | | | | | | | |
| Hair Sheen | 2 | 9 | 3 | 9 | 6 | 5 | 6 | 5 | 6 | 2 | 8 | 5.55 |
| and Luster | | | | | | | | | | | | |
| Static | 3 | 8 | 8 | 9 | 8 | 7 | 9 | 3 | 3 | 4 | 2 | 5.82 |
| Flyaways | | | | | | | | | | | | |
| Feel and | 4 | 7 | 10 | 5 | 4 | 8 | 3 | 8 | 2 | 8 | 9 | 6.18 |
| Touch of Dry | | | | | | | | | | | | |
| Hair | | | | | | | | | | | | |
| Overall | 1 | 7 | 2 | 10 | 4 | 9 | 4 | 2 | 9 | 4 | 10 | 5.64 |
| Condition of | | | | | | | | | | | | |
| Hair | | | | | | | | | | | | |

TABLE 5
Shampoo C Without Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 4 | 6 | 9 | 6 | 5 | 5 | 8 | 6 | 10 | 8 | 8 | 6.82 |
| Product | 10 | 7 | 6 | 9 | 10 | 5 | 10 | 7 | 9 | 6 | 6 | 7.73 |
| Fragrance | | | | | | | | | | | | |
| Pourability | 6 | 7 | 7 | 4 | 8 | 8 | 10 | 6 | 8 | 5 | 8 | 7.00 |
| Application | 4 | 10 | 8 | 4 | 7 | 5 | 6 | 6 | 10 | 7 | 9 | 6.91 |
| Ease | | | | | | | | | | | | |
| Fragrance at | 6 | 10 | 5 | 4 | 9 | 7 | 7 | 6 | 10 | 10 | 7 | 7.36 |
| Application | | | | | | | | | | | | |
| Foam Quality | 8 | 7 | 10 | 8 | 10 | 6 | 8 | 7 | 5 | 6 | 5 | 7.27 |
| Foam | 9 | 4 | 8 | 9 | 5 | 8 | 9 | 9 | 5 | 5 | 9 | 7.27 |
| Quantity | | | | | | | | | | | | |
| Fragrance | 1 | 5 | 5 | 6 | 1 | 1 | 3 | 1 | 6 | 2 | 7 | 3.45 |
| during | | | | | | | | | | | | |
| Drying | | | | | | | | | | | | |
| Ease of Wet | 4 | 3 | 2 | 5 | 3 | 7 | 3 | 7 | 6 | 1 | 3 | 4.00 |
| Combability | | | | | | | | | | | | |
| Ease of Dry | 4 | 5 | 3 | 6 | 4 | 2 | 2 | 7 | 2 | 5 | 7 | 4.27 |
| Combability | | | | | | | | | | | | |
| Residual | 6 | 7 | 1 | 6 | 1 | 5 | 5 | 2 | 3 | 3 | 5 | 4.00 |
| Fragrance on | | | | | | | | | | | | |
| Hair | | | | | | | | | | | | |
| Hair Sheen | 6 | 6 | 2 | 5 | 6 | 7 | 7 | 5 | 2 | 3 | 3 | 4.73 |
| and Luster | | | | | | | | | | | | |
| Static | 6 | 3 | 7 | 2 | 6 | 4 | 6 | 6 | 2 | 5 | 4 | 4.64 |
| Flyaways | | | | | | | | | | | | |
| Feel and | 4 | 3 | 1 | 4 | 4 | 6 | 3 | 7 | 7 | 7 | 3 | 4.45 |
| Touch of Dry | | | | | | | | | | | | |
| Hair | | | | | | | | | | | | |
| Overall | 1 | 6 | 2 | 1 | 6 | 5 | 2 | 5 | 2 | 6 | 1 | 3.36 |
| Condition of | | | | | | | | | | | | |
| Hair | | | | | | | | | | | | |

TABLE 6
Shampoo C With Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 8 | 8 | 10 | 8 | 4 | 10 | 5 | 4 | 5 | 9 | 6 | 7.00 |
| Product | 4 | 4 | 7 | 5 | 5 | 9 | 8 | 4 | 9 | 9 | 7 | 6.45 |
| Fragrance | | | | | | | | | | | | |
| Pourability | 6 | 7 | 9 | 8 | 10 | 4 | 5 | 9 | 9 | 9 | 4 | 7.27 |
| Application | 7 | 6 | 6 | 5 | 7 | 10 | 6 | 6 | 8 | 5 | 9 | 6.82 |
| Ease | | | | | | | | | | | | |
| Fragrance at | 4 | 10 | 10 | 4 | 5 | 4 | 10 | 8 | 8 | 5 | 10 | 7.09 |
| Application | | | | | | | | | | | | |
| Foam Quality | 9 | 8 | 8 | 9 | 4 | 4 | 9 | 10 | 5 | 5 | 8 | 7.18 |
| Foam | 5 | 5 | 7 | 10 | 7 | 8 | 7 | 8 | 4 | 9 | 8 | 7.09 |
| Quantity | | | | | | | | | | | | |
| Fragrance | 1 | 4 | 6 | 4 | 7 | 10 | 5 | 3 | 2 | 8 | 5 | 5.00 |
| during | | | | | | | | | | | | |
| Drying | | | | | | | | | | | | |
| Ease of Wet | 3 | 2 | 5 | 5 | 6 | 1 | 1 | 9 | 1 | 4 | 8 | 4.09 |
| Combability | | | | | | | | | | | | |
| Ease of Dry | 2 | 3 | 1 | 4 | 2 | 5 | 1 | 10 | 10 | 8 | 9 | 5.00 |
| Combability | | | | | | | | | | | | |
| Residual | 10 | 9 | 2 | 8 | 3 | 7 | 2 | 3 | 4 | 8 | 7 | 5.73 |
| Fragrance on | | | | | | | | | | | | |
| Hair | | | | | | | | | | | | |
| Hair Sheen | 6 | 2 | 10 | 2 | 7 | 8 | 3 | 10 | 7 | 8 | 3 | 6.00 |
| and Luster | | | | | | | | | | | | |
| Static | 5 | 1 | 4 | 9 | 5 | 2 | 2 | 8 | 2 | 8 | 4 | 4.55 |
| Flyaways | | | | | | | | | | | | |
| Feel and | 8 | 5 | 10 | 7 | 9 | 7 | 3 | 8 | 6 | 8 | 1 | 6.55 |
| Touch of Dry | | | | | | | | | | | | |
| Hair | | | | | | | | | | | | |
| Overall | 10 | 9 | 7 | 3 | 6 | 6 | 4 | 1 | 9 | 8 | 2 | 5.91 |
| Condition of | | | | | | | | | | | | |
| Hair | | | | | | | | | | | | |

Tables 7 through 12 summarize the ratings given by each hairstylist to each conditioner, both with and without the mineral composition. The hairstylists rated each conditioner according to several characteristics on a scale of 1 to 10, 10 being the best rating. The conditioners containing the mineral composition also exhibited enhanced performance. These conditioners enhanced the sheen and luster of the hair, enhanced the conditioning, moisturizing and hydrating of the hair, and reduced static flyaways.

TABLE 7
Conditioner A Without Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 8 | 4 | 10 | 10 | 5 | 7 | 5 | 1 | 8 | 5 | 8 | 7.27 |
| Product | 8 | 7 | 5 | 10 | 10 | 7 | 5 | 9 | 5 | 5 | 5 | 6.91 |
| Fragrance | | | | | | | | | | | | |
| Pourability | 6 | 6 | 10 | 4 | 9 | 9 | 4 | 9 | 10 | 8 | 9 | 7.64 |
| Application | 7 | 4 | 10 | 8 | 9 | 6 | 10 | 6 | 6 | 8 | 9 | 7.55 |
| Ease | | | | | | | | | | | | |
| Fragrance at | 5 | 7 | 6 | 9 | 9 | 10 | 10 | 8 | 4 | 6 | 7 | 7.36 |
| Application | | | | | | | | | | | | |
| Foam Quality | 10 | 5 | 10 | 7 | 8 | 10 | 4 | 10 | 5 | 6 | 8 | 7.55 |
| Foam | 7 | 7 | 8 | 4 | 10 | 7 | 7 | 6 | 6 | 10 | 4 | 6.91 |
| Quantity | | | | | | | | | | | | |
| Fragrance | 7 | 5 | 4 | 1 | 2 | 6 | 4 | 5 | 4 | 1 | 1 | 3.64 |
| during | | | | | | | | | | | | |
| Drying | | | | | | | | | | | | |
| Ease of Wet | 2 | 2 | 5 | 7 | 5 | 2 | 4 | 2 | 5 | 2 | 3 | 3.55 |
| Combability | | | | | | | | | | | | |
| Ease of Dry | 5 | 2 | 7 | 2 | 5 | 4 | 2 | 5 | 6 | 4 | 7 | 4.45 |
| Combability | | | | | | | | | | | | |
| Residual | 6 | 3 | 7 | 4 | 1 | 6 | 6 | 5 | 2 | 1 | 4 | 4.09 |
| Fragrance on | | | | | | | | | | | | |
| Hair | | | | | | | | | | | | |
| Hair Sheen | 1 | 7 | 2 | 3 | 4 | 2 | 3 | 1 | 2 | 2 | 4 | 2.82 |
| and Luster | | | | | | | | | | | | |
| Static | 6 | 7 | 1 | 2 | 2 | 7 | 4 | 6 | 5 | 3 | 6 | 4.45 |
| Flyaways | | | | | | | | | | | | |
| Feel and | 6 | 6 | 6 | 2 | 3 | 3 | 3 | 1 | 5 | 7 | 4 | 4.18 |
| Touch of Dry | | | | | | | | | | | | |
| Hair | | | | | | | | | | | | |
| Overall | 7 | 6 | 3 | 6 | 1 | 7 | 4 | 4 | 1 | 3 | 7 | 4.45 |
| Condition of | | | | | | | | | | | | |
| Hair | | | | | | | | | | | | |

TABLE 8
Conditioner A With Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 5 | 8 | 9 | 4 | 7 | 7 | 4 | 4 | 10 | 7 | 9 | 6.73 |
| Product | 10 | 9 | 9 | 6 | 7 | 8 | 10 | 10 | 6 | 8 | 7 | 8.18 |
| Fragrance | | | | | | | | | | | | |
| Pourability | 4 | 7 | 4 | 5 | 9 | 9 | 5 | 9 | 9 | 8 | 10 | 7.18 |
| Application | 5 | 6 | 4 | 4 | 9 | 4 | 9 | 9 | 8 | 4 | 7 | 6.27 |
| Ease | | | | | | | | | | | | |
| Fragrance at | 6 | 6 | 7 | 6 | 5 | 10 | 5 | 8 | 8 | 10 | 10 | 7.36 |
| Application | | | | | | | | | | | | |
| Foam Quality | 8 | 5 | 9 | 10 | 9 | 9 | 7 | 4 | 7 | 4 | 4 | 6.91 |
| Foam | 5 | 10 | 5 | 7 | 4 | 9 | 4 | 4 | 10 | 4 | 5 | 6.09 |
| Quantity | | | | | | | | | | | | |
| Fragrance | 6 | 10 | 7 | 3 | 7 | 1 | 1 | 9 | 6 | 4 | 7 | 5.55 |
| during | | | | | | | | | | | | |
| Drying | | | | | | | | | | | | |
| Ease of Wet | 1 | 3 | 1 | 1 | 6 | 1 | 1 | 2 | 9 | 4 | 9 | 3.45 |
| Combability | | | | | | | | | | | | |
| Ease of Dry | 4 | 8 | 10 | 6 | 2 | 9 | 3 | 7 | 5 | 7 | 10 | 6.45 |
| Combability | | | | | | | | | | | | |
| Residual | 1 | 7 | 10 | 4 | 7 | 2 | 7 | 10 | 7 | 5 | 8 | 6.18 |
| Fragrance on | | | | | | | | | | | | |
| Hair | | | | | | | | | | | | |
| Hair Sheen | 5 | 5 | 9 | 4 | 3 | 9 | 6 | 4 | 8 | 8 | 2 | 5.73 |
| and Luster | | | | | | | | | | | | |
| Static | 7 | 6 | 1 | 7 | 9 | 7 | 1 | 5 | 2 | 8 | 9 | 5.64 |
| Flyaways | | | | | | | | | | | | |
| Feel and | 8 | 3 | 10 | 1 | 4 | 7 | 4 | 10 | 7 | 10 | 1 | 5.91 |
| Touch of Dry | | | | | | | | | | | | |
| Hair | | | | | | | | | | | | |
| Overall | 3 | 6 | 2 | 2 | 9 | 6 | 6 | 2 | 6 | 7 | 7 | 5.09 |
| Condition of | | | | | | | | | | | | |
| Hair | | | | | | | | | | | | |

TABLE 9
Conditioner B Without Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 6 | 9 | 5 | 8 | 5 | 4 | 4 | 9 | 10 | 9 | 10 | 7.18 |
| Product | 6 | 7 | 4 | 8 | 7 | 10 | 4 | 5 | 5 | 9 | 10 | 6.82 |
| Fragrance | | | | | | | | | | | | |
| Pourability | 9 | 10 | 8 | 7 | 7 | 5 | 6 | 10 | 4 | 5 | 4 | 6.82 |
| Application | 9 | 4 | 4 | 8 | 9 | 4 | 4 | 4 | 4 | 5 | 9 | 5.82 |
| Ease | | | | | | | | | | | | |
| Fragrance at | 10 | 8 | 8 | 6 | 6 | 5 | 10 | 10 | 9 | 4 | 7 | 7.55 |
| Application | | | | | | | | | | | | |
| Foam Quality | 5 | 5 | 6 | 9 | 6 | 6 | 8 | 6 | 6 | 7 | 9 | 6.64 |
| Foam | 4 | 8 | 10 | 7 | 6 | 4 | 6 | 6 | 7 | 4 | 9 | 6.45 |
| Quantity | | | | | | | | | | | | |
| Fragrance | 5 | 5 | 6 | 7 | 3 | 6 | 6 | 1 | 4 | 7 | 4 | 4.91 |
| during | | | | | | | | | | | | |
| Drying | | | | | | | | | | | | |
| Ease of Wet | 5 | 5 | 2 | 1 | 6 | 1 | 4 | 6 | 3 | 4 | 5 | 3.82 |
| Combability | | | | | | | | | | | | |
| Ease of Dry | 6 | 4 | 2 | 4 | 3 | 4 | 1 | 4 | 6 | 4 | 7 | 4.09 |
| Combability | | | | | | | | | | | | |
| Residual | 7 | 2 | 5 | 4 | 4 | 1 | 1 | 7 | 3 | 4 | 7 | 4.09 |
| Fragrance on | | | | | | | | | | | | |
| Hair | | | | | | | | | | | | |
| Hair Sheen | 2 | 1 | 2 | 6 | 4 | 7 | 3 | 2 | 2 | 5 | 1 | 3.18 |
| and Luster | | | | | | | | | | | | |
| Static | 7 | 4 | 6 | 6 | 6 | 7 | 7 | 2 | 6 | 6 | 4 | 5.55 |
| Flyaways | | | | | | | | | | | | |
| Feel and | 5 | 3 | 7 | 6 | 5 | 3 | 3 | 3 | 5 | 1 | 6 | 4.27 |
| Touch of Dry | | | | | | | | | | | | |
| Hair | | | | | | | | | | | | |
| Overall | 4 | 2 | 5 | 2 | 6 | 4 | 7 | 3 | 7 | 3 | 4 | 4.27 |
| Condition of | | | | | | | | | | | | |
| Hair | | | | | | | | | | | | |

TABLE 10
Conditioner B With Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 8 | 10 | 9 | 5 | 5 | 7 | 4 | 10 | 7 | 4 | 4 | 6.64 |
| Product | 10 | 5 | 10 | 10 | 7 | 7 | 8 | 8 | 4 | 10 | 4 | 7.55 |
| Fragrance | | | | | | | | | | | | |
| Pourability | 8 | 4 | 4 | 8 | 8 | 7 | 8 | 4 | 4 | 5 | 5 | 5.91 |
| Application | 10 | 8 | 9 | 4 | 8 | 10 | 10 | 10 | 8 | 4 | 9 | 8.18 |
| Ease | | | | | | | | | | | | |
| Fragrance at | 6 | 9 | 4 | 8 | 7 | 4 | 9 | 7 | 9 | 5 | 4 | 6.55 |
| Application | | | | | | | | | | | | |
| Foam Quality | 5 | 9 | 6 | 4 | 5 | 4 | 7 | 9 | 4 | 4 | 10 | 6.09 |
| Foam | 8 | 8 | 9 | 7 | 5 | 10 | 8 | 10 | 4 | 8 | 10 | 7.91 |
| Quantity | | | | | | | | | | | | |
| Fragrance | 8 | 10 | 8 | 3 | 2 | 10 | 7 | 10 | 1 | 7 | 3 | 6.27 |
| during | | | | | | | | | | | | |
| Drying | | | | | | | | | | | | |
| Ease of Wet | 8 | 1 | 9 | 4 | 4 | 10 | 9 | 3 | 4 | 10 | 10 | 6.55 |
| Combability | | | | | | | | | | | | |
| Ease of Dry | 7 | 3 | 4 | 1 | 9 | 9 | 7 | 3 | 2 | 8 | 2 | 5.00 |
| Combability | | | | | | | | | | | | |
| Residual | 8 | 6 | 4 | 1 | 2 | 7 | 7 | 8 | 6 | 8 | 6 | 5.73 |
| Fragrance on | | | | | | | | | | | | |
| Hair | | | | | | | | | | | | |
| Hair Sheen | 2 | 9 | 3 | 9 | 6 | 5 | 6 | 5 | 6 | 2 | 8 | 5.55 |
| and Luster | | | | | | | | | | | | |
| Static | 3 | 8 | 8 | 9 | 8 | 7 | 9 | 3 | 3 | 4 | 2 | 5.82 |
| Flyaways | | | | | | | | | | | | |
| Feel and | 4 | 7 | 10 | 5 | 4 | 8 | 3 | 8 | 2 | 8 | 9 | 6.18 |
| Touch of Dry | | | | | | | | | | | | |
| Hair | | | | | | | | | | | | |
| Overall | 1 | 7 | 2 | 10 | 4 | 9 | 4 | 2 | 9 | 4 | 10 | 5.64 |
| Condition of | | | | | | | | | | | | |
| Hair | | | | | | | | | | | | |

TABLE 11
Conditioner C Without Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 4 | 6 | 9 | 6 | 5 | 5 | 8 | 6 | 10 | 8 | 8 | 6.82 |
| Product | 10 | 7 | 6 | 9 | 10 | 5 | 10 | 7 | 9 | 6 | 6 | 7.73 |
| Fragrance | | | | | | | | | | | | |
| Pourability | 6 | 7 | 7 | 4 | 8 | 8 | 10 | 6 | 8 | 5 | 8 | 7.00 |
| Application | 4 | 10 | 8 | 4 | 7 | 5 | 6 | 6 | 10 | 7 | 9 | 6.91 |
| Ease | | | | | | | | | | | | |
| Fragrance at | 6 | 10 | 5 | 4 | 9 | 7 | 7 | 6 | 10 | 10 | 7 | 7.36 |
| Application | | | | | | | | | | | | |
| Foam Quality | 8 | 7 | 10 | 8 | 10 | 6 | 8 | 7 | 5 | 6 | 5 | 7.27 |
| Foam | 9 | 4 | 8 | 9 | 5 | 8 | 9 | 9 | 5 | 5 | 9 | 7.27 |
| Quantity | | | | | | | | | | | | |
| Fragrance | 1 | 5 | 5 | 6 | 1 | 1 | 3 | 1 | 6 | 2 | 7 | 3.45 |
| during | | | | | | | | | | | | |
| Drying | | | | | | | | | | | | |
| Ease of Wet | 4 | 3 | 2 | 5 | 3 | 7 | 3 | 7 | 6 | 1 | 3 | 4.00 |
| Combability | | | | | | | | | | | | |
| Ease of Dry | 4 | 5 | 3 | 6 | 4 | 2 | 2 | 7 | 2 | 5 | 7 | 4.27 |
| Combability | | | | | | | | | | | | |
| Residual | 6 | 7 | 1 | 6 | 1 | 5 | 5 | 2 | 3 | 3 | 5 | 4.00 |
| Fragrance on | | | | | | | | | | | | |
| Hair | | | | | | | | | | | | |
| Hair Sheen | 6 | 6 | 2 | 5 | 6 | 7 | 7 | 5 | 2 | 3 | 3 | 4.73 |
| and Luster | | | | | | | | | | | | |
| Static | 6 | 3 | 7 | 2 | 6 | 4 | 6 | 6 | 2 | 5 | 4 | 4.64 |
| Flyaways | | | | | | | | | | | | |
| Feel and | 4 | 3 | 1 | 4 | 4 | 6 | 3 | 7 | 7 | 7 | 3 | 4.45 |
| Touch of Dry | | | | | | | | | | | | |
| Hair | | | | | | | | | | | | |
| Overall | 1 | 6 | 2 | 1 | 6 | 5 | 2 | 5 | 2 | 6 | 1 | 3.36 |
| Condition of | | | | | | | | | | | | |
| Hair | | | | | | | | | | | | |

TABLE 12
Conditioner C With Mineral Composition Added

| Test | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Attributes | #1 | #2 | #3 | #4 | #5 | #6 | #7 | #8 | #9 | #10 | #11 | Ratings |
| Appearance | 8 | 8 | 10 | 8 | 4 | 10 | 5 | 4 | 5 | 9 | 6 | 7.00 |
| Product | 4 | 4 | 7 | 5 | 5 | 9 | 8 | 4 | 9 | 9 | 7 | 6.45 |
| Fragrance | | | | | | | | | | | | |
| Pourability | 6 | 7 | 9 | 8 | 10 | 4 | 5 | 9 | 9 | 9 | 4 | 7.27 |
| Application | 7 | 6 | 6 | 5 | 7 | 10 | 6 | 6 | 8 | 5 | 9 | 6.82 |
| Ease | | | | | | | | | | | | |
| Fragrance at | 4 | 10 | 10 | 4 | 5 | 4 | 10 | 8 | 8 | 5 | 10 | 7.09 |
| Application | | | | | | | | | | | | |
| Foam Quality | 9 | 8 | 8 | 9 | 4 | 4 | 9 | 10 | 5 | 5 | 8 | 7.18 |
| Foam | 5 | 5 | 7 | 10 | 7 | 8 | 7 | 8 | 4 | 9 | 8 | 7.09 |
| Quantity | | | | | | | | | | | | |
| Fragrance | 1 | 4 | 6 | 4 | 7 | 10 | 5 | 3 | 2 | 8 | 5 | 5.00 |
| during | | | | | | | | | | | | |
| Drying | | | | | | | | | | | | |
| Ease of Wet | 3 | 2 | 5 | 5 | 6 | 1 | 1 | 9 | 1 | 4 | 8 | 4.09 |
| Combability | | | | | | | | | | | | |
| Ease of Dry | 2 | 3 | 1 | 4 | 2 | 5 | 1 | 10 | 10 | 8 | 9 | 5.00 |
| Combability | | | | | | | | | | | | |
| Residual | 10 | 9 | 2 | 8 | 3 | 7 | 2 | 3 | 4 | 8 | 7 | 5.73 |
| Fragrance on | | | | | | | | | | | | |
| Hair | | | | | | | | | | | | |
| Hair Sheen | 6 | 2 | 10 | 2 | 7 | 8 | 3 | 10 | 7 | 8 | 3 | 6.00 |
| and Luster | | | | | | | | | | | | |
| Static | 5 | 1 | 4 | 9 | 5 | 2 | 2 | 8 | 2 | 8 | 4 | 4.55 |
| Flyaways | | | | | | | | | | | | |
| Feel and | 8 | 5 | 10 | 7 | 9 | 7 | 3 | 8 | 6 | 8 | 1 | 6.55 |
| Touch of Dry | | | | | | | | | | | | |
| Hair | | | | | | | | | | | | |
| Overall | 10 | 9 | 7 | 3 | 6 | 6 | 4 | 1 | 9 | 8 | 2 | 5.91 |
| Condition of | | | | | | | | | | | | |
| Hair | | | | | | | | | | | | |

Hair conditioners were also separately tested for moisture retention by treating three separate swatches of hair with three different conditioners, placing them in a dehydration chamber and weighing the swatches at regular time intervals. The test was also conducted on three similar swatches treated with conditioners not containing the mineral composition. Tables 13 through 18 summarize the results of these tests, showing that the conditioners containing the mineral composition exhibit substantially enhanced moisture retention.

TABLE 13
Water Retention of Conditioner D With Mineral
Composition Added

| Time Elapsed | Swatch #1 | Swatch #2 | Swatch #3 | Average |
|---|---|---|---|---|
| (minutes) | (grams) | (grams) | (grams) | (grams) |
| 0 | 2.38 | 2.70 | 2.78 | 2.62 |
| 15 | 2.33 | 2.66 | 2.69 | 2.56 |
| 30 | 2.31 | 2.53 | 2.55 | 2.46 |
| 45 | 2.26 | 2.41 | 2.46 | 2.38 |
| 60 | 2.19 | 2.35 | 2.39 | 2.31 |
| 75 | 2.15 | 2.26 | 2.32 | 2.24 |
| 90 | 2.04 | 2.22 | 2.37 | 2.21 |

The results reported in Table 13 show that after 90 minutes in the dehydration chamber Swatch No. 1 lost approximately 14.29% of its weight, Swatch No. 2 lost approximately 17.78% of its weight, and Swatch No. 3 lost approximately 14.75% of its weight. The average weight loss after 90 minutes in the dehydration chamber was approximately 15.65%.

TABLE 14
Water Retention of Conditioner D Without Mineral
Composition Added

| Time | Swatch #1 | Swatch #2 | Swatch #3 | |
|---|---|---|---|---|
| Elapsed (minutes) | (grams) | (grams) | (grams) | Average (grams) |
| 0 | 2.63 | 2.49 | 2.89 | 2.67 |
| 15 | 2.51 | 2.42 | 2.67 | 2.53 |
| 30 | 2.41 | 2.36 | 2.58 | 2.45 |
| 45 | 2.38 | 2.29 | 2.53 | 2.40 |
| 60 | 2.25 | 2.13 | 2.41 | 2.26 |
| 75 | 2.01 | 2.05 | 2.08 | 2.05 |
| 90 | 1.89 | 1.87 | 1.93 | 1.90 |

The results reported in Table 14 show that after 90 minutes in the dehydration chamber Swatch No. 1 lost approximately 28.14% of its weight, Swatch No. 2 lost approximately 24.90% of its weight, and Swatch No. 3 lost approximately 33.22% of its weight. The average weight loss after 90 minutes in the dehydration chamber was approximately 28.96%, a substantially greater percentage than that of the conditioners containing the mineral composition.

TABLE 15
Water Retention of Conditioner E With Mineral
Composition Added

| Time | Swatch #1 | Swatch #2 | Swatch #3 | |
|---|---|---|---|---|
| Elapsed (minutes) | (grams) | (grams) | (grams) | Average (grams) |
| 0 | 2.58 | 2.89 | 3.02 | 2.83 |
| 15 | 2.56 | 2.84 | 2.81 | 2.74 |
| 30 | 2.51 | 2.76 | 2.79 | 2.69 |
| 45 | 2.46 | 2.71 | 2.73 | 2.63 |
| 60 | 2.55 | 2.68 | 2.69 | 2.64 |
| 75 | 2.35 | 2.56 | 2.65 | 2.52 |
| 90 | 2.22 | 2.45 | 2.63 | 2.43 |

The results reported in Table 15 show that after 90 minutes in the dehydration chamber Swatch No. 1 lost approximately 13.95% of its weight, Swatch No. 2 lost approximately 15.22% of its weight, and Swatch No. 3 lost approximately 12.91% of its weight. The average weight loss after 90 minutes in the dehydration chamber was approximately 14.02%.

TABLE 16
Water Retention of Conditioner E Without Mineral
Composition Added

| Time Elapsed | Swatch #1 | Swatch #2 | Swatch #3 | |
|---|---|---|---|---|
| (minutes) | (grams) | (grams) | (grams) | Average (grams) |
| 0 | 2.84 | 2.56 | 3.21 | 2.87 |
| 15 | 2.66 | 2.42 | 3.07 | 2.72 |
| 30 | 2.53 | 2.36 | 2.95 | 2.61 |
| 45 | 2.36 | 2.28 | 2.84 | 2.49 |
| 60 | 2.28 | 2.23 | 2.78 | 2.43 |
| 75 | 2.25 | 2.18 | 2.72 | 2.38 |
| 90 | 2.22 | 2.05 | 2.63 | 2.30 |

The results reported in Table 16 show that after 90 minutes in the dehydration chamber Swatch No. 1 lost approximately 21.83% of its weight, Swatch No. 2 lost approximately 19.92% of its weight, and Swatch No. 3 lost approximately 18.07% of its weight. The average weight loss after 90 minutes in the dehydration chamber was approximately 19.86%, a substantially greater percentage than that of the conditioners containing the mineral composition.

TABLE 17
Water Retention of Conditioner F With Mineral
Composition Added

| Time | Swatch #1 | Swatch #2 | Swatch #3 | |
|---|---|---|---|---|
| Elapsed (minutes) | (grams) | (grams) | (grams) | Average (grams) |
| 0 | 1.99 | 2.21 | 2.40 | 2.20 |
| 15 | 1.86 | 2.18 | 2.35 | 2.13 |
| 30 | 1.86 | 2.17 | 2.24 | 2.09 |
| 45 | 1.84 | 2.13 | 2.18 | 2.05 |
| 60 | 1.84 | 1.99 | 1.93 | 1.92 |
| 75 | 1.80 | 1.97 | 2.11 | 1.96 |
| 90 | 1.74 | 1.95 | 2.15 | 1.95 |

The results reported in Table 17 show that after 90 minutes in the dehydration chamber Swatch No. 1 lost approximately 12.56% of its weight, Swatch No. 2 lost approximately 11.76% of its weight, and Swatch No. 3 lost approximately 10.42% of its weight. The average weight loss after 90 minutes in the dehydration chamber was approximately 11.52%.

TABLE 18
Water Retention of Conditioner F Without Mineral
Composition Added

| Time | Swatch #1 | Swatch #2 | Swatch #3 | |
|---|---|---|---|---|
| Elapsed (minutes) | (grams) | (grams) | (grams) | Average (grams) |
| 0 | 2.15 | 2.01 | 1.98 | 2.05 |
| 15 | 1.99 | 1.85 | 1.78 | 1.87 |
| 30 | 2.05 | 1.76 | 1.71 | 1.84 |
| 45 | 1.87 | 1.72 | 1.69 | 1.76 |
| 60 | 1.64 | 1.69 | 1.67 | 1.67 |
| 75 | 1.70 | 1.71 | 1.66 | 1.69 |
| 90 | 1.78 | 1.69 | 1.62 | 1.70 |

The results reported in Table 18 show that after 90 minutes in the dehydration chamber Swatch No. 1 lost approximately 17.21% of its weight, Swatch No. 2 lost approximately 15.92% of its weight, and Swatch No. 3 lost approximately 18.18% of its weight. The average weight loss after 90 minutes in the dehydration chamber was approximately 17.10%, a substantially greater percentage than that of the conditioners containing the mineral composition.

Tables 19 through 24 summarize the ratings given by each hairstylist to each hairspray, both with and without the mineral composition. The hairstylists rated each hairspray according to several characteristics on a scale of 1 to 10, 10 being the best rating. The hairsprays containing the mineral composition also exhibited enhanced performance. These hairsprays provided enhanced style hold, enhanced gloss, reduced static flyaways and enhanced ease of combing or brushing.

TABLE 19
Hairspray A Without Mineral Composition Added

| | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Test Attributes | #1 | #2 | #3 | #4 | #5 | #6 | #7 | #8 | #9 | #10 | #11 | Ratings |
| Spray | 3 | 6 | 5 | 6 | 5 | 5 | 3 | 4 | 4 | 6 | 4 | 4.64 |
| Characteristics | | | | | | | | | | | | |
| Target Coverage | 7 | 4 | 4 | 7 | 3 | 4 | 4 | 4 | 3 | 4 | 5 | 4.45 |
| Workability | 3 | 6 | 3 | 7 | 7 | 5 | 7 | 6 | 5 | 5 | 5 | 5.36 |
| Drying Time | 7 | 6 | 4 | 7 | 3 | 7 | 4 | 5 | 4 | 7 | 4 | 5.27 |
| Hold | 4 | 4 | 4 | 4 | 4 | 3 | 5 | 4 | 6 | 3 | 3 | 4.00 |
| Sheen | 3 | 4 | 5 | 7 | 3 | 3 | 6 | 5 | 5 | 3 | 6 | 4.55 |
| Stylability | 3 | 4 | 4 | 4 | 6 | 3 | 5 | 6 | 5 | 4 | 5 | 4.45 |
| Post Style | 6 | 5 | 3 | 5 | 4 | 5 | 6 | 4 | 5 | 5 | 3 | 4.64 |
| Handling | | | | | | | | | | | | |
| Style Memory | 5 | 4 | 6 | 6 | 7 | 4 | 3 | 4 | 5 | 7 | 3 | 4.91 |
| Flaking | 4 | 3 | 3 | 6 | 3 | 5 | 7 | 5 | 4 | 5 | 7 | 4.73 |
| Static Flyaway | 5 | 3 | 3 | 7 | 7 | 5 | 3 | 6 | 7 | 7 | 3 | 5.09 |
| Overall | 6 | 7 | 7 | 5 | 6 | 7 | 6 | 3 | 5 | 7 | 4 | 5.73 |
| Condition of | | | | | | | | | | | | |
| Hair | | | | | | | | | | | | |

TABLE 20
Hairspray A With Mineral Composition Added

| | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Test Attributes | #1 | #2 | #3 | #4 | #5 | #6 | #7 | #8 | #9 | #10 | #11 | Ratings |
| Spray | 7 | 5 | 6 | 6 | 5 | 3 | 5 | 7 | 6 | 3 | 3 | 5.09 |
| Characteristics | | | | | | | | | | | | |
| Target Coverage | 7 | 7 | 4 | 3 | 5 | 4 | 7 | 6 | 4 | 6 | 5 | 5.27 |
| Workability | 5 | 4 | 7 | 6 | 7 | 7 | 5 | 7 | 7 | 6 | 5 | 6.00 |
| Drying Time | 7 | 7 | 6 | 6 | 8 | 5 | 8 | 5 | 7 | 8 | 8 | 6.82 |
| Hold | 5 | 6 | 7 | 8 | 7 | 7 | 5 | 7 | 8 | 7 | 7 | 6.73 |
| Sheen | 6 | 6 | 7 | 5 | 6 | 8 | 7 | 7 | 6 | 5 | 8 | 6.45 |
| Stylability | 8 | 6 | 7 | 7 | 7 | 8 | 6 | 5 | 6 | 8 | 7 | 6.82 |
| Post Style | 7 | 8 | 7 | 7 | 7 | 7 | 7 | 5 | 5 | 7 | 5 | 6.55 |
| Handling | | | | | | | | | | | | |
| Style Memory | 8 | 8 | 7 | 6 | 7 | 7 | 5 | 6 | 8 | 7 | 6 | 6.82 |
| Flaking | 8 | 5 | 6 | 6 | 5 | 8 | 7 | 7 | 7 | 8 | 7 | 6.73 |
| Static Flyaway | 6 | 8 | 5 | 8 | 6 | 8 | 5 | 6 | 5 | 8 | 7 | 6.55 |
| Overall | 8 | 5 | 7 | 5 | 5 | 6 | 6 | 8 | 8 | 5 | 8 | 6.45 |
| Condition of | | | | | | | | | | | | |
| Hair | | | | | | | | | | | | |

TABLE 21
Hairspray B Without Mineral Composition Added

| | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Test Attributes | #1 | #2 | #3 | #4 | #5 | #6 | #7 | #8 | #9 | #10 | #11 | Ratings |
| Spray | 6 | 3 | 7 | 6 | 5 | 3 | 6 | 5 | 3 | 4 | 4 | 4.73 |
| Characteristics | | | | | | | | | | | | |
| Target Coverage | 6 | 3 | 7 | 6 | 5 | 7 | 5 | 3 | 3 | 3 | 4 | 4.73 |
| Workability | 5 | 4 | 3 | 4 | 5 | 3 | 3 | 4 | 5 | 4 | 6 | 4.18 |
| Drying Time | 7 | 5 | 6 | 7 | 3 | 3 | 4 | 3 | 4 | 3 | 5 | 4.55 |
| Hold | 4 | 3 | 4 | 4 | 7 | 4 | 5 | 4 | 7 | 7 | 7 | 5.09 |
| Sheen | 7 | 6 | 6 | 6 | 3 | 5 | 4 | 6 | 4 | 4 | 7 | 5.27 |
| Stylability | 5 | 5 | 4 | 4 | 6 | 5 | 5 | 5 | 3 | 7 | 7 | 5.09 |
| Post Style | 5 | 7 | 3 | 3 | 6 | 3 | 7 | 6 | 4 | 6 | 7 | 5.18 |
| Handling | | | | | | | | | | | | |
| Style Memory | 4 | 5 | 5 | 5 | 4 | 7 | 5 | 7 | 7 | 7 | 6 | 5.64 |
| Flaking | 6 | 3 | 6 | 4 | 4 | 7 | 5 | 4 | 7 | 3 | 5 | 4.91 |
| Static Flyaway | 6 | 7 | 7 | 5 | 5 | 6 | 6 | 4 | 7 | 6 | 7 | 6.00 |
| Overall | 7 | 5 | 6 | 4 | 3 | 5 | 6 | 4 | 3 | 4 | 4 | 4.64 |
| Condition of | | | | | | | | | | | | |
| Hair | | | | | | | | | | | | |

TABLE 22
Hairspray B With Mineral Composition Added

| | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Test Attributes | #1 | #2 | #3 | #4 | #5 | #6 | #7 | #8 | #9 | #10 | #11 | Ratings |
| Spray | 7 | 7 | 7 | 5 | 7 | 3 | 6 | 7 | 4 | 6 | 5 | 5.82 |
| Characteristics | | | | | | | | | | | | |
| Target Coverage | 3 | 4 | 4 | 7 | 3 | 3 | 6 | 7 | 7 | 5 | 5 | 4.91 |
| Workability | 6 | 3 | 3 | 3 | 7 | 4 | 7 | 3 | 7 | 3 | 3 | 4.45 |
| Drying Time | 6 | 8 | 7 | 8 | 7 | 6 | 7 | 8 | 6 | 6 | 7 | 6.91 |
| Hold | 8 | 7 | 8 | 8 | 7 | 7 | 5 | 8 | 7 | 7 | 8 | 7.27 |
| Sheen | 5 | 8 | 6 | 6 | 8 | 6 | 8 | 7 | 5 | 6 | 7 | 6.55 |
| Stylability | 5 | 8 | 6 | 7 | 6 | 5 | 5 | 8 | 8 | 8 | 5 | 6.45 |
| Post Style | 8 | 7 | 8 | 5 | 5 | 6 | 5 | 8 | 6 | 5 | 5 | 6.18 |
| Handling | | | | | | | | | | | | |
| Style Memory | 8 | 7 | 6 | 7 | 6 | 8 | 5 | 6 | 7 | 6 | 5 | 6.45 |
| Flaking | 8 | 8 | 6 | 7 | 6 | 8 | 7 | 8 | 6 | 7 | 7 | 7.09 |
| Static Flyaway | 8 | 8 | 5 | 6 | 6 | 6 | 8 | 6 | 6 | 5 | 6 | 6.36 |
| Overall | 6 | 5 | 8 | 8 | 5 | 5 | 5 | 6 | 8 | 8 | 8 | 6.64 |
| Condition of | | | | | | | | | | | | |
| Hair | | | | | | | | | | | | |

TABLE 23
Hairspray C Without Mineral Composition Added

| | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Test Attributes | #1 | #2 | #3 | #4 | #5 | #6 | #7 | #8 | #9 | #10 | #11 | Ratings |
| Spray | 4 | 6 | 7 | 5 | 5 | 6 | 5 | 5 | 5 | 4 | 7 | 5.36 |
| Characteristics | | | | | | | | | | | | |
| Target Coverage | 7 | 5 | 4 | 3 | 4 | 3 | 5 | 5 | 6 | 4 | 3 | 4.45 |
| Workability | 4 | 6 | 7 | 4 | 4 | 5 | 3 | 7 | 3 | 5 | 5 | 4.82 |
| Drying Time | 6 | 7 | 4 | 4 | 6 | 3 | 4 | 6 | 5 | 7 | 4 | 5.09 |
| Hold | 3 | 6 | 4 | 6 | 5 | 6 | 4 | 7 | 3 | 7 | 7 | 5.27 |
| Sheen | 6 | 3 | 5 | 5 | 7 | 4 | 6 | 6 | 6 | 6 | 3 | 5.18 |
| Stylability | 4 | 7 | 7 | 7 | 6 | 4 | 7 | 3 | 7 | 3 | 6 | 5.55 |
| Post Style | 3 | 6 | 5 | 7 | 7 | 7 | 6 | 6 | 4 | 7 | 5 | 5.73 |
| Handling | | | | | | | | | | | | |
| Style Memory | 3 | 7 | 5 | 6 | 7 | 6 | 3 | 5 | 6 | 5 | 5 | 5.27 |
| Flaking | 4 | 5 | 5 | 4 | 7 | 7 | 3 | 5 | 7 | 7 | 7 | 5.55 |
| Static Flyaway | 7 | 3 | 6 | 3 | 3 | 5 | 3 | 4 | 5 | 3 | 5 | 4.27 |
| Overall | 4 | 7 | 6 | 3 | 4 | 5 | 4 | 4 | 4 | 6 | 3 | 4.55 |
| Condition of | | | | | | | | | | | | |
| Hair | | | | | | | | | | | | |

TABLE 24
Hairspray C With Mineral Composition Added

| | H.S. | H.S. | H.S. | H.S. | H.S. | H.S. | H.S. | H.S. | H.S. | H.S. | H.S. | Average |
|---|---|---|---|---|---|---|---|---|---|---|---|---|
| Test Attributes | #1 | #2 | #3 | #4 | #5 | #6 | #7 | #8 | #9 | #10 | #11 | Ratings |
| Spray | 7 | 3 | 5 | 7 | 6 | 4 | 7 | 4 | 6 | 3 | 7 | 5.36 |
| Characteristics | | | | | | | | | | | | |
| Target Coverage | 4 | 7 | 7 | 4 | 3 | 6 | 7 | 6 | 4 | 7 | 5 | 5.45 |
| Workability | 3 | 6 | 7 | 3 | 7 | 7 | 4 | 5 | 7 | 7 | 3 | 5.36 |
| Drying Time | 8 | 5 | 8 | 7 | 8 | 6 | 5 | 6 | 6 | 6 | 7 | 6.55 |
| Hold | 5 | 6 | 7 | 5 | 5 | 5 | 7 | 5 | 6 | 8 | 7 | 6.00 |
| Sheen | 8 | 8 | 5 | 6 | 5 | 6 | 5 | 7 | 5 | 8 | 5 | 6.18 |
| Stylability | 6 | 6 | 6 | 7 | 8 | 5 | 5 | 6 | 5 | 7 | 7 | 6.18 |
| Post Style | 7 | 8 | 8 | 7 | 5 | 8 | 5 | 6 | 6 | 5 | 8 | 6.64 |
| Handling | | | | | | | | | | | | |
| Style Memory | 8 | 7 | 5 | 8 | 6 | 6 | 8 | 8 | 8 | 5 | 8 | 7.00 |
| Flaking | 7 | 8 | 5 | 5 | 7 | 8 | 6 | 6 | 6 | 7 | 5 | 6.36 |
| Static Flyaway | 7 | 8 | 6 | 8 | 6 | 7 | 6 | 8 | 8 | 7 | 6 | 7.00 |
| Overall | 6 | 7 | 7 | 6 | 5 | 5 | 8 | 6 | 6 | 7 | 5 | 6.18 |
| Condition of | | | | | | | | | | | | |
| Hair | | | | | | | | | | | | |

Hairsprays were also separately tested for curl retention under extremely humid conditions. Three separate swatches of hair were treated with three different hairsprays, curled around a glass rod with a diameter of about 1 cm and allowed to air dry. The lengths between the first and last loops of the curled swatches were recorded, and the swatches were then placed in a humidity chamber measuring 90% relative humidity. The distance between the first and last loops of the curled swatches were recorded at regular time intervals. The test was also conducted on three separate swatches treated with hairsprays not containing the mineral composition. Tables 25 through 30 summarize the results of this testing, showing that the hairsprays containing the mineral composition exhibit enhanced curl retention.

TABLE 25
Curl Retention of Hairspray D Without Mineral
Composition Added

| Time | Length of | Length of Hair | Length of Hair | Percent |
|---|---|---|---|---|
| Elapsed | Hair Before | Before Exposure | After Exposure | Curl |
| (minutes) | Curled | to Humidity | to Humidity | Retention |
| 0 | 8.00 | 4.20 | 4.20 | 100.00 |
| 30 | 8.00 | 4.20 | 4.53 | 91.32 |
| 60 | 8.00 | 4.20 | 4.88 | 82.11 |
| 90 | 8.00 | 4.20 | 4.61 | 89.21 |
| 120 | 8.00 | 4.20 | 5.01 | 78.68 |
| 150 | 8.00 | 4.20 | 5.25 | 72.37 |
| 180 | 8.00 | 4.20 | 5.55 | 64.47 |

TABLE 26
Curl Retention of Hairspray D With Mineral
Composition Added

| Time | Length of | Length of Hair | Length of Hair | Percent |
|---|---|---|---|---|
| Elapsed | Hair Before | Before Exposure | After Exposure | Curl |
| (minutes) | Curled | to Humidity | to Humidity | Retention |
| 0 | 8.00 | 4.53 | 4.53 | 100.00 |
| 30 | 8.00 | 4.53 | 4.89 | 89.63 |
| 60 | 8.00 | 4.53 | 4.66 | 96.25 |
| 90 | 8.00 | 4.53 | 4.88 | 89.91 |
| 120 | 8.00 | 4.53 | 4.92 | 88.76 |
| 150 | 8.00 | 4.53 | 5.01 | 86.17 |
| 180 | 8.00 | 4.53 | 5.05 | 85.01 |

TABLE 27
Curl Retention of Hairspray E Without Mineral
Composition Added

| Time | Length of | Length of Hair | Length of Hair | Percent |
|---|---|---|---|---|
| Elapsed | Hair Before | Before Exposure | After Exposure | Curl |
| (minutes) | Curled | to Humidity | to Humidity | Retention |
| 0 | 8.00 | 6.20 | 6.20 | 100.00 |
| 30 | 8.00 | 6.20 | 6.49 | 83.89 |
| 60 | 8.00 | 6.20 | 6.62 | 76.67 |
| 90 | 8.00 | 6.20 | 6.83 | 65.00 |
| 120 | 8.00 | 6.20 | 6.88 | 62.22 |
| 150 | 8.00 | 6.20 | 6.93 | 59.44 |
| 180 | 8.00 | 6.20 | 7.15 | 47.22 |

TABLE 28
Curl Retention of Hairspray E With Mineral
Composition Added

| Time | Length of | Length of Hair | Length of Hair | Percent |
|---|---|---|---|---|
| Elapsed | Hair Before | Before Exposure | After Exposure | Curl |
| (minutes) | Curled | to Humidity | to Humidity | Retention |
| 0 | 8.00 | 6.58 | 6.58 | 100.00 |
| 30 | 8.00 | 6.58 | 6.73 | 89.44 |
| 60 | 8.00 | 6.58 | 6.72 | 90.14 |
| 90 | 8.00 | 6.58 | 6.80 | 84.51 |
| 120 | 8.00 | 6.58 | 6.75 | 88.03 |
| 150 | 8.00 | 6.58 | 7.00 | 70.42 |
| 180 | 8.00 | 6.58 | 7.20 | 56.34 |

TABLE 29
Curl Retention of Hairspray F Without Mineral
Composition Added

| | | Length of Hair | Length of Hair | |
|---|---|---|---|---|
| Time Elapsed | Length of Hair | Before Exposure | After Exposure to | Percent Curl |
| (minutes) | Before Curled | to Humidity | Humidity | Retention |
| 0 | 8.00 | 5.89 | 5.89 | 100.00 |
| 30 | 8.00 | 5.89 | 6.20 | 85.31 |
| 60 | 8.00 | 5.89 | 6.12 | 89.10 |
| 90 | 8.00 | 5.89 | 6.35 | 78.20 |
| 120 | 8.00 | 5.89 | 6.49 | 71.56 |
| 150 | 8.00 | 5.89 | 6.58 | 67.30 |
| 180 | 8.00 | 5.89 | 6.79 | 57.35 |

TABLE 30
Curl Retention of Hairspray F Without Mineral
Composition Added

| | | Length of Hair | Length of Hair | |
|---|---|---|---|---|
| Time Elapsed | Length of Hair | Before Exposure | After Exposure to | Percent Curl |
| (minutes) | Before Curled | to Humidity | Humidity | Retention |
| 0 | 8.00 | 6.35 | 6.35 | 100.00 |
| 30 | 8.00 | 6.35 | 6.39 | 97.58 |
| 60 | 8.00 | 6.35 | 6.49 | 91.52 |
| 90 | 8.00 | 6.35 | 6.56 | 87.27 |
| 120 | 8.00 | 6.35 | 6.72 | 77.58 |
| 150 | 8.00 | 6.35 | 6.71 | 78.18 |
| 180 | 8.00 | 6.35 | 6.76 | 75.15 |

Tables 25 through 30 show an average curl retention of 56.35% after 180 minutes in the humidity chamber for swatches treated with hairsprays not containing the mineral composition. The average curl retention of swatches treated with hairspray containing the mineral composition after 180 minutes in the humidity chamber is 72.17%. Therefore, the hairsprays containing the mineral composition exhibit substantially greater curl retention than those not containing the mineral composition.

The preceding description has been presented with reference to presently preferred embodiments of the invention. Workers skilled in the art and technology to which this invention pertains will appreciate that alterations and changes in the described embodiments may be practiced without meaningfully departing from the principal, spirit and scope of this invention.

Accordingly, the foregoing description should not be read as pertaining only to the precise embodiments described, but rather should be read consistent with and as support for the following claims which are to have their fullest and fairest scope.

1. A mineral composition for use in hair care products comprising at least one poly-element mineral selected from the group consisting of perlite, pitchstone and tourmaline, ground into a powder having a particle size ranging from about 0.5 μm to about 10 μm.

2. A mineral composition according to claim 1, wherein the powder has a particle size ranging from about 0.5 μm to about 5.0 μm.

3. A mineral composition according to claim 1, wherein the powder has a particle size ranging from about 0.5 μm to about 2.5 μm.

4. A mineral composition according to claim 1, wherein the powder has a particle size ranging from about 0.5 μm to about 1.0 μm.

5. A mineral composition according to claim 1, wherein the at least one poly-element mineral comprises at least two poly-element minerals.

6. A mineral composition according to claim 5, wherein the at least two poly-element minerals are tourmaline and perlite, wherein tourmaline is present in an amount ranging from about 0.50% to about 99.5% by weight, with perlite making up the remaining wt %.

7. A mineral composition according to claim 6, wherein tourmaline is present in an amount ranging from 0.50% to about 50% by weight, with perlite making up the remaining wt %.

8. A mineral composition according to claim 6, wherein tourmaline is present in an amount of about 0.50% by weight, and perlite is present in an amount of about 99.5% by weight.

9. A mineral composition according to claim 5, wherein the at least two poly-element minerals are tourmaline and pitchstone, wherein tourmaline is present in amount ranging from about 0.50% to about 99.5% by weight, with pitchstone making up the remaining wt %.

10. A mineral composition according to claim 9, wherein tourmaline is present in an amount ranging from about 0.50% to about 50% by weight, with pitchstone making up the remaining wt %.

11. A mineral composition according to claim 9, wherein tourmaline is present in an amount of about 0.50% by weight, and pitchstone is present in an amount of about 99.5% by weight.

12. A mineral composition according to claim 5, wherein the at least two poly-element minerals are perlite and pitchstone, wherein perlite is present in an amount ranging from about 0.50% to about 99.5% by weight, with pitchstone making up the remaining wt %.

13. A mineral composition according to claim 12, wherein perlite is present in an amount ranging from about 0.50% to about 50%, with pitchstone making up the remaining wt %.

14. A mineral composition according to claim 12, wherein perlite is present in an amount of about 0.50% by weight, and pitchstone is present in an amount of about 99.5% by weight.

15. A mineral composition according to claim 1, wherein the at least one poly-element mineral comprises all three poly-element minerals, wherein tourmaline is present in an amount ranging from about 0.10% to about 99.8% by weight, and perlite is present in an amount ranging from about 0.10% to about 99.8% by weight, with pitchstone making up the remaining wt %.

16. A mineral composition according to claim 15, wherein tourmaline is present in an amount of about 0.10% by weight, perlite is present in an amount of about 0.10% by weight, and pitchstone is present in an amount of about 99.8% by weight.

17. A mineral composition according to claim 15, wherein tourmaline is present in an amount of about 0.10% by weight, perlite is present in an amount of about 99.8% by weight, and pitchstone is present in an amount of about 0.10% by weight.

18. A mineral composition according to claim 15, wherein tourmaline is present in an amount of about 99.8%, perlite is present in an amount of about 0.10%, and pitchstone is present in an amount of about 0.10%.

19. A mineral composition for use in hair care products comprising at least one poly-element mineral ground to a powder having a particle size ranging from about 0.05 mm to about 25 mm and suspended in a solution with a compatible solvent.

20. A mineral composition according to claim 19, wherein the solvent is selected from the group consisting of deionized water and glycol or its derivatives.

21. A mineral composition according to claim 19, wherein the at least one poly-element mineral is ground to a powder having a particle size ranging from about 0.05 mm to about 10 mm.

22. A mineral composition according to claim 19, wherein the at least one poly-element mineral is ground to a powder having a particle size ranging from about 0.05 mm to about 5 mm.

23. A mineral composition according to claim 19, wherein the at least one poly-element mineral is ground to a powder having a particle size ranging from about 0.05 mm to about 0.1 mm.

24. A method of preparing an ionic mineral composition for use in hair care products comprising:
preparing the mineral composition of claim 19; and
heating the suspended solution under pressure for a suitable length of time.

25. A method according to claim 24, wherein the compatible solvent is selected from the group consisting of deionized water and glycol or its derivatives.

26. A method according to claim 24, wherein the suspended solution is heated for a period of time ranging from about 12 to about 120 hours.

27. A method according to claim 24, wherein the suspended solution is heated for a period of time ranging from about 24 to about 48 hours.

28. A method according to claim 24, wherein the compatible solvent is deionized water.

29. A method according to claim 28, wherein the suspended solution is heated at a temperature ranging from about 5° C. to about 100° C.

30. A method according to claim 28, wherein the suspended solution is heated at a temperature ranging from about 30° C. to about 80° C.

31. A method according to claim 28, wherein the suspended solution is heated at a temperature ranging from about 50° C. to about 60° C.

32. A method according to claim 24, wherein the suspended solution is heated under a pressure ranging from about 1 atm to about 5 atm.

33. A method according to claim 24, wherein the suspended solution is heated under a pressure ranging from about 1 atm to about 2 atm.

34. A method according to claim 24, wherein the suspended solution is heated under a pressure ranging from about 1 atm to about 1.25 atm.

35. A method according to claim 24, wherein the weight ratio of the powder of the at least one poly-element mineral to the compatible solvent ranges from about 0.05:1 to about 2.5:1.

36. A method according to claim 24, wherein the weight ratio of the powder of the at least one poly-element mineral to the solvent ranges from about 0.5:1 to about 1:1.

37. A method according to claim 24, wherein the compatible solvent is glycol or one of its derivatives.

38. A method according to claim 37, wherein the suspended solution is heated at a temperature ranging from about 75° C. to about 200° C.

39. A method according to claim 37, wherein the suspended solution is heated at a temperature ranging from about 150° C. to about 200° C.

40. A method according to claim 37, wherein the suspended solution is heated at a temperature ranging from about 100° C. to about 150° C.

41. A method according to claim 37, wherein the suspended solution is heated at a temperature ranging from about 75° C. to about 100° C.

42. A method of preparing an ionic mineral composition for use in hair care products comprising:
preparing the mineral composition of claim 20, wherein the compatible solvent is glycol or one of its derivatives; and
heating the suspended solution at a temperature ranging from about 75° C. to about 100° C. under pressure ranging from about 1 atm to about 1.25 atm for a period of time ranging from about 24 to about 48 hours.

43. A method of preparing an ionic mineral composition for use in hair care products comprising:
preparing the mineral composition of claim 20, wherein the compatible solvent is deionized water; and
heating the suspended solution at a temperature ranging from 50° C. to 60° C. under a pressure ranging from 1 atm to 1.25 atm for a period of time ranging from 24 to 48 hours.

44. A method of applying a mineral composition for use in hair care products to the hair comprising:
preparing the mineral composition of claim 19;
adding the mineral composition to a hair care product;
thoroughly mixing the mineral composition into the hair care product; and
applying the hair care product to the hair.

45. A method of applying a mineral composition for use in hair care products to the hair comprising:
preparing the mineral composition of claim 1;
adding the mineral composition to a hair care product;
thoroughly mixing the mineral composition into the hair care product; and
applying the hair care product to the hair.

46. A method according to claim 45, wherein the mineral composition is present in the hair care product in an amount ranging from about 0.01% to about 10% by weight, based on the total weight of the product.

47. A method according to claim 45, wherein the mineral composition is present in the hair care product in an amount ranging from about 0.01% to about 5% by weight, based on the total weight of the product.

48. A method according to claim 45, wherein the mineral composition is present in the hair care product in an amount ranging from about 0.01% to about 0.5% by weight, based on the total weight of the product.

49. A method according to claim 45, wherein the mineral composition is present in the hair care product in an amount ranging from 0.01% to about 0.1% by weight, based on the total weight of the product.

50. A method according to claim 44, wherein the mineral composition is present in the hair care product in an amount ranging from about 0.01% to about 10% by weight, based on the total weight of the product.

51. A method according to claim 44, wherein the mineral composition is present in the hair care product in an amount ranging from about 0.01% to about 5% by weight, based on the total weight of the product.

52. A method according to claim 44, wherein the mineral composition is present in the hair care product in an amount ranging from about 0.01% to about 0.5% by weight, based on the total weight of the product.

53. A method according to claim 44, wherein the mineral composition is present in the hair care product in an amount ranging from about 0.01% to about 0.1% by weight, based on the total weight of the product.

54. A mineral composition for use in hair care products comprising tourmaline and perlite, wherein tourmaline is present in an amount of about 0.5% by weight, and perlite is present in an amount of about 99.5% by weight.
